(19) United States
(12) Patent Application Publication
(10) Pub. No.: US 2024/0315543 A1
(43) Pub. Date: Sep. 26, 2024

(54) REPROCESSING CONFIRMATION METHOD FOR ENDOSCOPE, REUSE PREVENTION TOOL, AND ENDOSCOPE

(71) Applicant: OLYMPUS MEDICAL SYSTEMS CORP., Tokyo (JP)

(72) Inventor: Yumiko AWAU, Tokyo (JP)

(73) Assignee: OLYMPUS MEDICAL SYSTEMS CORP., Tokyo (JP)

(21) Appl. No.: 18/679,121

(22) Filed: May 30, 2024

Related U.S. Application Data
(63) Continuation of application No. PCT/JP2022/020534, filed on May 17, 2022.

(51) Int. Cl. A61B 1/12; A61B 1/00

(52) U.S. Cl. CPC A61B 1/123; A61B 1/00059

(57) ABSTRACT

A reprocessing confirmation method for an endoscope by a processor includes: recording an attachment state of a reuse prevention tool of the endoscope before shipping to which the reuse prevention tool is attached; after the endoscope is returned, comparing the attachment state of the reuse prevention tool with the attachment state recorded, before removing the reuse prevention tool for reprocessing and further outputting a comparison result; and recording the attachment state of the reuse prevention tool of the endoscope before shipping to which the reuse prevention tool is newly attached after reprocessing.

### CROSS REFERENCE TO RELATED APPLICATION

This application is a continuation application of PCT/JP2022/020534 filed on May 17, 2022, the entire contents of which are incorporated herein by this reference.

### BACKGROUND OF THE INVENTION

### 1. Field of the Invention

The present disclosure relates to a reprocessing confirmation method for an endoscope, a reuse prevention tool, and an endoscope in a rental service for the endoscope.

### 2. Description of the Related Art

In recent years, a rental service renting out an endoscope to a hospital or the like has been known. In such a rental service of the endoscope, it is necessary to prevent the endoscope from being wrongly used by a rental destination such as a hospital.

For example, Japanese Patent Application Laid-Open Publication No. 2009-160312 discloses a technique in which if the number of connecting times and a connecting time period between an endoscope and a processor, or the like have exceeded a specified value, a signal route for picking up an image of the endoscope is blocked through a network. In this manner, a method for restricting the use of the endoscope has been well known in which the use of the endoscope is disabled by utilizing information-communication technology.

In a rental service of an endoscope, a rental source company rents out a reprocessed endoscope to a hospital or the like. Then, a rental service company collects and reprocesses the endoscope after use.

In particular, in an endoscope for medical use, reprocessing is important to prevent a medical accident such as a secondary infection from occurring. Therefore, the rental source company performs reprocessing at a stage at which a used endoscope is returned to a reprocessing center or the like.

### SUMMARY OF THE INVENTION

In a reprocessing confirmation method for an endoscope according to an aspect of the present disclosure, a processor is configured to: record an attachment state of a reuse prevention tool of the endoscope before shipping to which the reuse prevention tool is attached; after the endoscope is returned, compare the attachment state of the reuse prevention tool with the attachment state recorded, before removing the reuse prevention tool for reprocessing and further output a comparison result; and record the attachment state of the reuse prevention tool of the endoscope before shipping to which the reuse prevention tool is newly attached after reprocessing.

A reuse prevention tool according to an aspect of the present disclosure includes: a cover that covers at least one part of an endoscope accessory or an endoscope main body portion on an endoscope surface, with a light guide exposed so as to be connectable to an endoscope processor; and a fixing portion that fixes the cover to the endoscope surface.

In an endoscope according to an aspect of the present disclosure, a reuse prevention tool, which includes a cover that covers at least one part of an endoscope accessory or an endoscope main body portion on an endoscope surface, with a light guide exposed so as to be connectable to an endoscope processor; and a fixing portion that fixes the cover to the endoscope surface, is disposed on an endoscope main body, the endoscope accessory, and a surface of the endoscope accessory or the endoscope main body portion.

### BRIEF DESCRIPTION OF THE DRAWINGS

FIG. 1 is a view showing a hospital and a reprocessing center in a rental business of an endoscope;

FIG. 2 is a view showing a configuration of the endoscope;

FIG. 3 is a view showing a state in which an air/water feeding button and a suction button are removed from an operation portion of the endoscope;

FIG. 4 is a view before a cleaning tube is attached to the endoscope;

FIG. 5 is a view showing a state in which the cleaning tube is attached to the endoscope;

FIG. 6 is a view showing a state before the cleaning tube and an air feeding tube are attached to a scope connector;

FIG. 7 is a view showing a state in which the cleaning tube and the air feeding tube are attached to the scope connector;

FIG. 8 is a view showing a state in which the endoscope is placed in a treatment tank of an endoscope reprocessor;

FIG. 9 is a view showing a state before the cleaning tube and the air feeding tube are attached to a scope connector in another form;

FIG. 10 is a view showing a state in which the endoscope including the scope connector is placed in the treatment tank of the endoscope reprocessor;

FIG. 11 is a perspective view showing the endoscope to which a first reuse prevention tool is attached;

FIG. 12 is a top view showing the endoscope to which the first reuse prevention tool is attached;

FIG. 13 is a side view showing the scope connector to which a second reuse prevention tool is attached;

FIG. 14 is a side view showing the scope connector to which a third reuse prevention tool is attached;

FIG. 15 is a side view showing the scope connector and a universal cord to which a fourth reuse prevention tool is attached;

FIG. 16 is a side view showing the universal cord and the operation portion to which a fifth reuse prevention tool is attached;

FIG. 17 is a flowchart explaining a reprocessing method for the endoscope; and

FIG. 18 is a flowchart for explaining a modification of a reprocessing confirmation method for the endoscope.

### DETAILED DESCRIPTION OF THE PREFERRED EMBODIMENT

Hereinafter, an endoscope for medical use as insertion equipment will be described as an example. Note that in the following descriptions, the drawings based on each embodiment are schematic illustrations, and it should be noted that the relation between the thickness and the width of each portion, the ratio in thickness of each portion, and the like differ from the actual relation, ratio, and the like, and there are also some portions with different dimensional relations and ratios among the drawings.

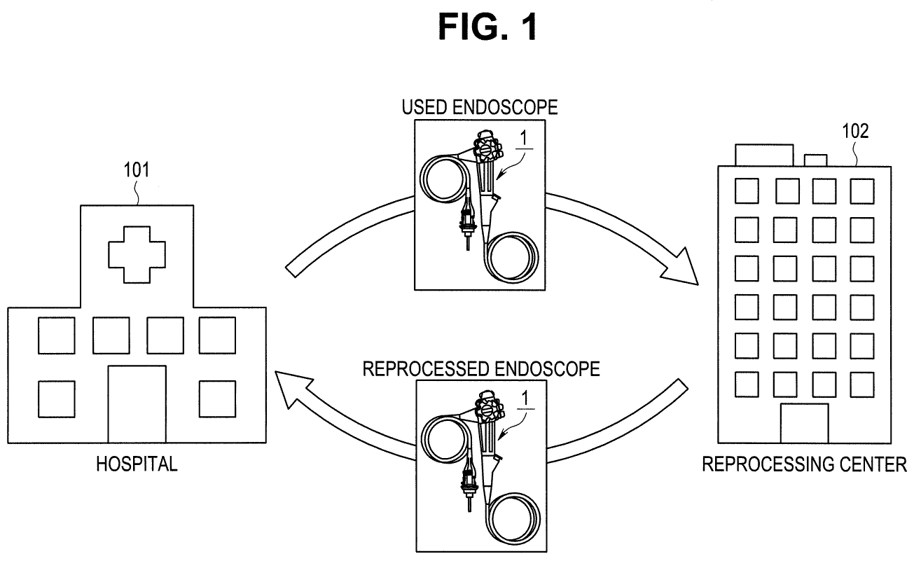

First, as shown in FIG. 1, the present embodiment is a technique applied to an endoscope rental business, in which a rental source company, which rents out an endoscope 1 that has been reprocessed to a hospital 101 or the like as a rental destination, collects and reprocesses the endoscope 1 that has been used, at a reprocessing center 102 so that the endoscope 1 is cleaned to be reusable.

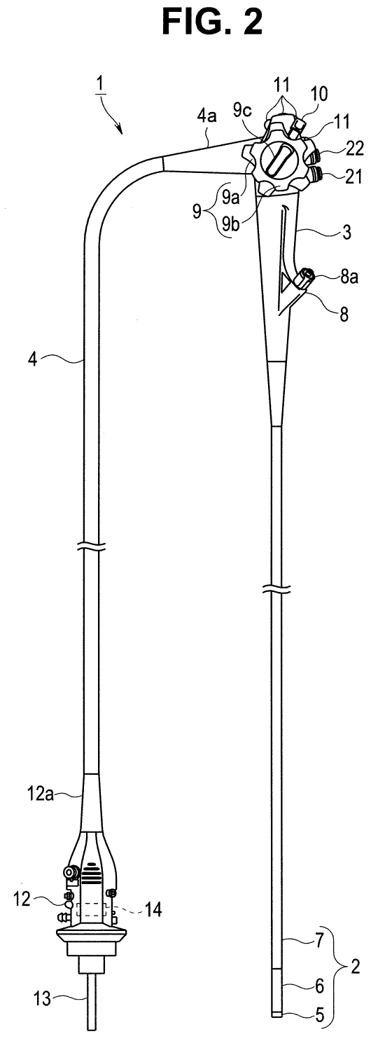

First, a schematic configuration of the endoscope 1 will be described. As shown in FIG. 2, the endoscope 1 includes an endoscope main body portion with an insertion portion 2, an operation portion 3, and a universal cord 4.

The insertion portion 2 is a long member insertable into a subject. The operation portion 3 is continuously provided at a proximal end of the insertion portion 2. The universal cord 4 is a composite cable extending from a side portion of the operation portion 3.

The insertion portion 2 includes a distal end portion 5, a bending portion 6, and a flexible tube portion 7 as a flexible tube. The distal end portion 5 is disposed at a distal end of the insertion portion 2. The bending portion 6 is actively bendable. The bending portion 6 is continuously provided on a proximal end side of the distal end portion 5. The flexible tube portion 7 is continuously provided on a proximal end side of the bending portion 6. The flexible tube portion 7 is connected so as to extend from a distal end of the operation portion 3.

The operation portion 3 includes a treatment instrument insertion opening 8, an angle knob 9, a UD angle fixing lever 10, a remote switch 11, an air/water feeding button 21, and a suction button 22.

The treatment instrument insertion opening 8 is an opening portion communicating with a treatment instrument insertion channel (not shown) as an endoscope conduit. The treatment instrument insertion opening 8 is provided with a forceps plug 8a as a forceps pipe sleeve.

The angle knob 9 is an operation handle for bending operation of the bending portion 6. The angle knob 9 includes a UD angle knob 9a and an RL angle knob 9b.

The UD angle knob 9a is an operation handle for bending operation of the bending portion 6 in an up-down direction. The RL angle knob 9b is an operation handle for bending operation of the bending portion 6 in a right-left direction. Note that an RL angle fixing knob 9c is disposed overlapping the RL angle knob 9b.

Four remote switches 11 are provided herein. The four remote switches 11 are operation switches for a video system and each of the operation switches is assigned for performing image halting, recording, switching of metering, enlargement of an image, or the like.

The air/water feeding button 21 is an operation button that controls delivery operation for fluid from a fluid delivery portion (not shown) provided in the distal end portion 5. For example, a structure that can perform air feeding by closing a small hole at the center of the air/water feeding button 21 with a finger, or the like, may be adopted. In addition, a structure that can perform water feeding by pressing the air/water feeding button 21, or the like, may be adopted.

The suction button 22 is an operation button that controls the inside of the treatment instrument insertion channel (not shown) to be under a negative pressure. For example, mucus or the like adhering to the distal end portion 5 can be sucked to be removed through an opening portion (not shown) of the treatment instrument insertion channel by pressing the suction button 22.

A bend preventing portion 4a is disposed in a contact portion between the universal cord 4 and the operation portion 3. A scope connector 12 of the endoscope main body portion is disposed on a proximal end side of the universal cord 4. The scope connector 12 includes a bend preventing portion 12a in a contact portion with the universal cord 4.

The scope connector 12 is connected to a video processor (not shown) that is an endoscope processor as an external apparatus including a light source device. A light guide connector 13 as well as an electric contact or the like is provided in such a manner as extending from the scope connector 12.

The video processor to which the scope connector 12 is connected transmits light emitted from an internal light source to a light guide bundle (not shown) inserted through the universal cord 4, the operation portion 3, and the insertion portion 2. Then, the light transmitted to the light guide bundle is emitted, as an illumination light, through an illumination window (not shown) provided in the distal end portion 5.

Note that the distal end portion 5 includes an observation window (not shown). The distal end portion 5 includes a built-in image sensor (not shown) that photoelectrically converts a light incident through the observation window. An image pickup cable extending from the image sensor is disposed in the scope connector 12 by insertion through the insertion portion 2, the operation portion 3, and the universal cord 4.

The scope connector 12 includes a built-in RF tag 14 such as an IC tag in which endoscope information is embedded. Note that with the use of an RFID reader that is available for a long-distance reading, endoscope information written in the RF tag 14 can be read from the outside of an endoscope case in which the endoscope 1 is packaged. Thus, the endoscope 1 includes the RF tag 14 constituting a part of an RFID system.

The endoscope 1 described above is a reuse product that is reprocessed for reuse. Therefore, the used endoscope 1 is transported to the reprocessing center 102 after being collected from the hospital 101.

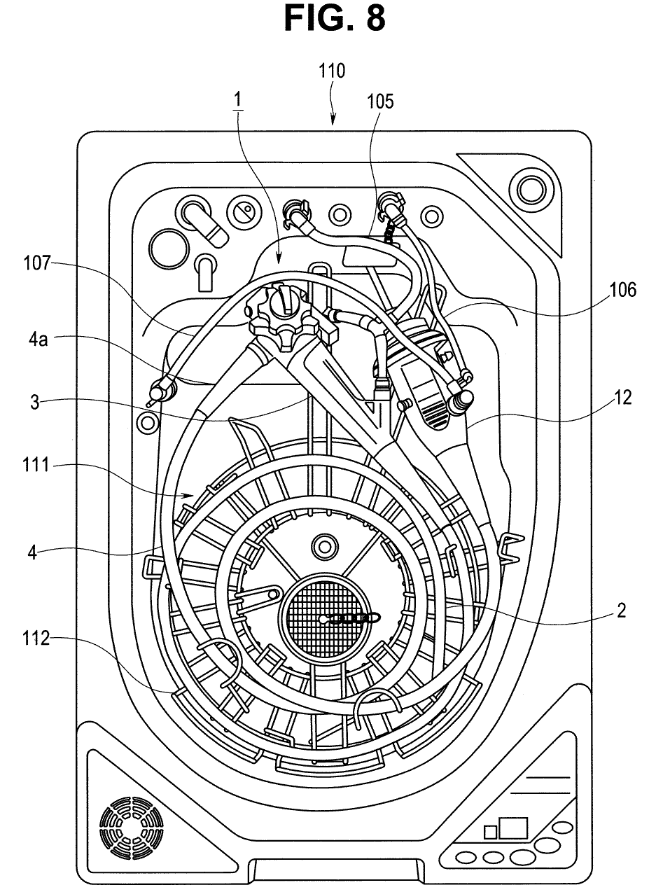

Then, the used endoscope 1 is reprocessed by an endoscope reprocessor 110 (see FIG. 8) at the reprocessing center 102 so as to be reusable. Note that since a configuration of the endoscope reprocessor 110 is publicly known, the details will be omitted.

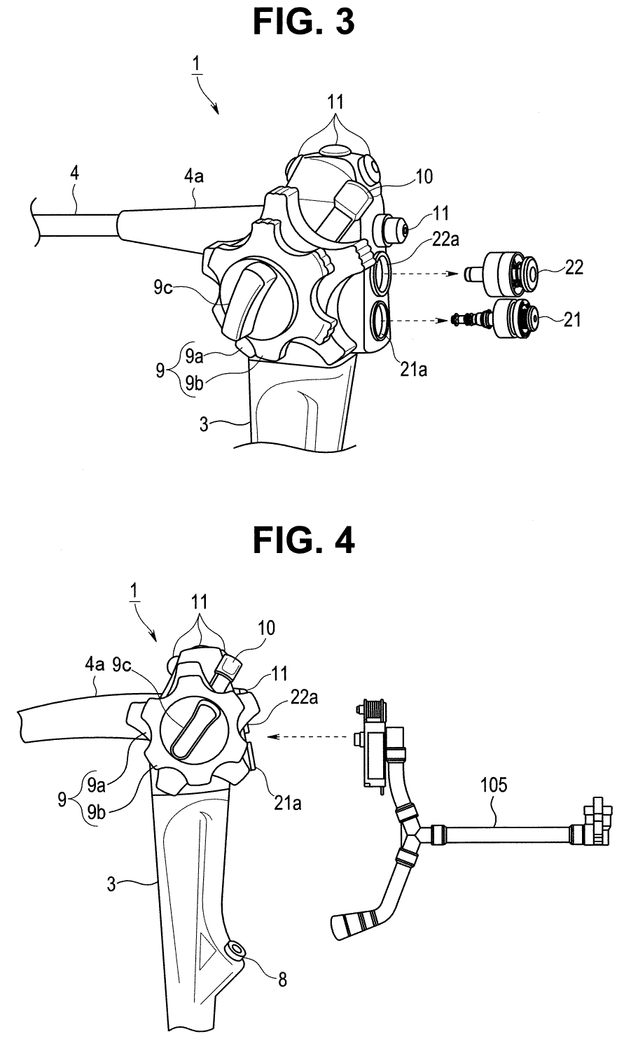

First, in reprocessing the endoscope 1, the air/water feeding button 21 and the suction button 22 of the operation portion 3 are removed, as shown in FIG. 3. The air/water feeding button 21 and the suction button 22 may be reusable or disposable.

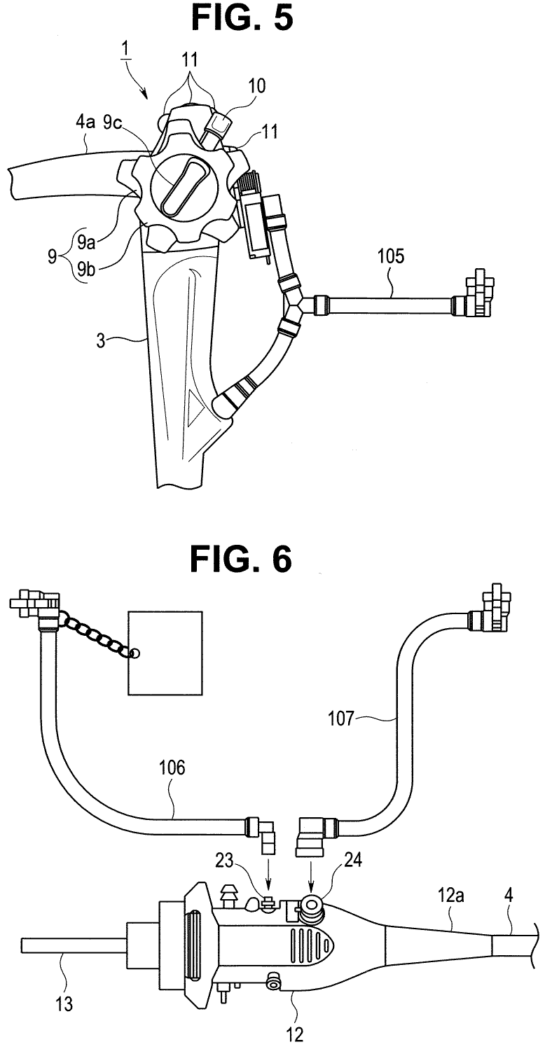

Then, in the operation portion 3, a cleaning tube 105 is connected to an air/water feeding cylinder 21a and a suction cylinder 22a from which the air/water feeding button 21 and the suction button 22 have been removed, as shown in FIG. 4 and FIG. 5.

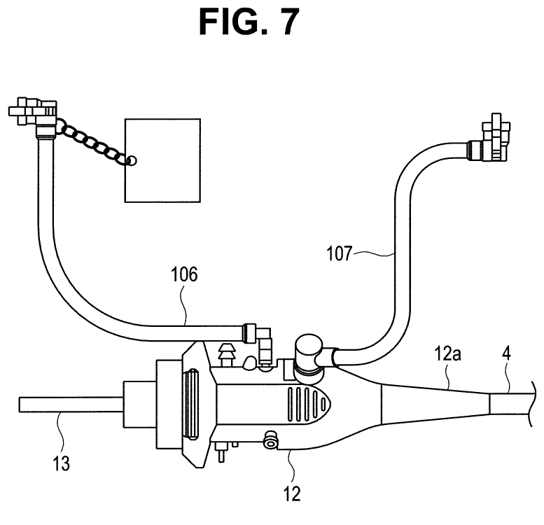

In the scope connector 12, a cleaning tube 106 is also connected to a pipe sleeve 23 for suction, sub-water feeding, or the like, as shown in FIG. 6 and FIG. 7. Further, in the scope connector 12, an air feeding tube 107 is connected to a ventilation pipe sleeve 24 for water leak testing.

The endoscope 1 is placed inside a treatment tank 111 of a cleaning tank of the endoscope reprocessor 110, as shown in FIG. 8. At this time, the endoscope 1, which is made of, for example, metal or resin, is set in a retaining net 112 attached to a bottom portion of the treatment tank 111. Note that in the treatment tank 111, the cleaning tubes 105, 106 and the air feeding tube 107 are connected such that the endoscope 1 and the endoscope reprocessor 110 communicate with each other.

In this manner, the endoscope 1 is placed inside the treatment tank 111 of the endoscope reprocessor 110 and is subjected to predetermined reprocessing.

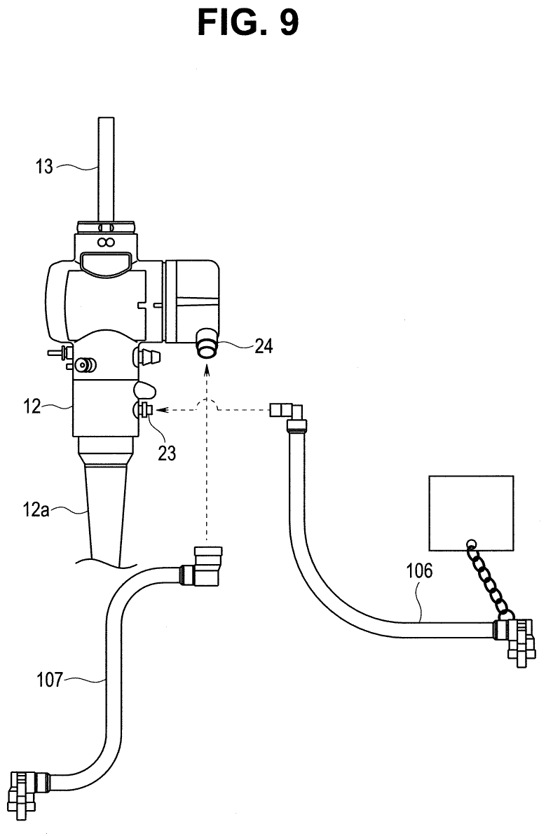

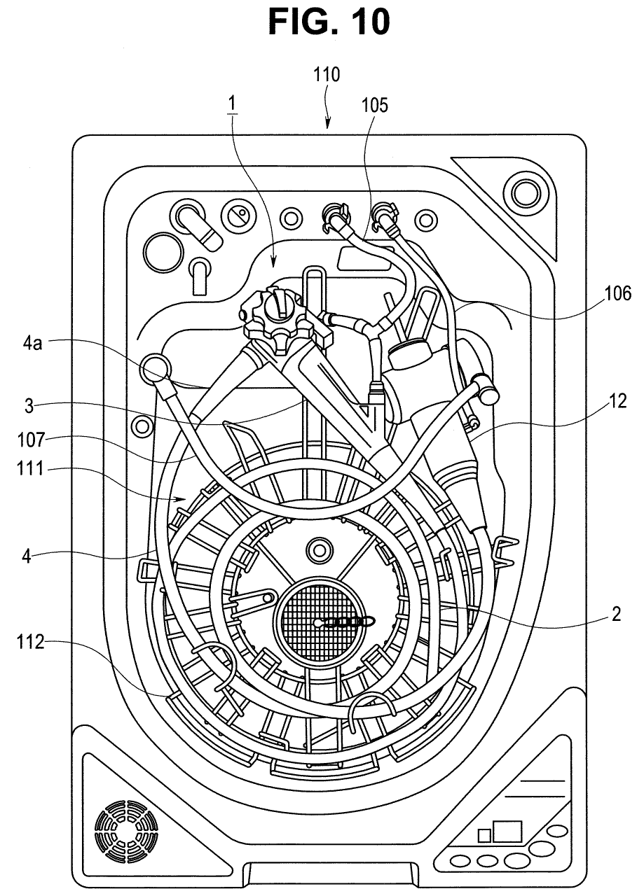

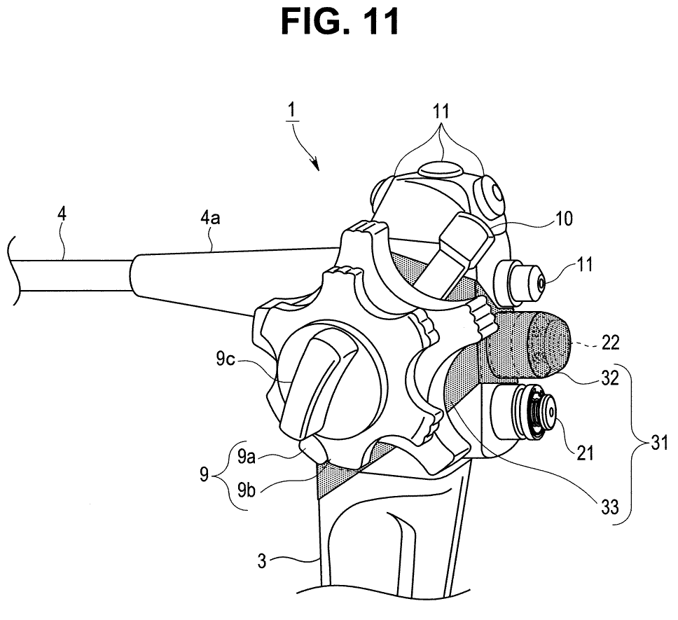

Note that the endoscope 1, depending on the model, includes a different type of scope connector 12 as shown in FIG. 9. In such a scope connector 12 also, the cleaning tube 106 is connected to the pipe sleeve 23 for suction, sub-water feeding, or the like, and the air feeding tube 107 is connected to the ventilation pipe sleeve 24 for water leak testing. Then, the endoscope 1 is set in the retaining net 112 and placed inside the treatment tank 111, as shown in FIG. 10.

In this manner, in reprocessing the endoscope 1 by the endoscope reprocessor 110, the cleaning tubes 105, 106 and the air feeding tube 107 are connected such that the endoscope 1 and the endoscope reprocessor 110 communicate with each other.

In the present embodiment, a reuse prevention tool for preventing the used endoscope 1 from being reused by the hospital 101 is attached to the endoscope 1. Note that a plurality of reuse prevention tools will be illustrated in the following description.

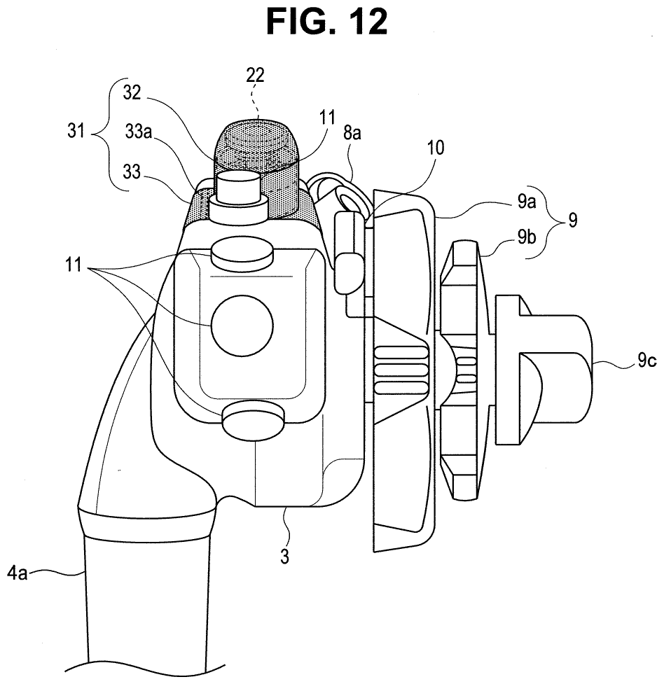

As shown in FIG. 11 and FIG. 12, a first reuse prevention tool 31 is configured as, for example, a removal prevention tool that prevents the suction button 22 of the endoscope accessory provided in the operation portion 3 from being removed from the suction cylinder 22a.

Specifically, the first reuse prevention tool 31 includes a cover portion 32 and a fixing portion 33 that fixes the cover portion 32 to the surface of the endoscope 1. The cover portion 32 is provided so as to cover the suction button 22. The cover portion 32 only needs to be able to prevent the suction button 22 from falling off and may cover the entire surface of the suction button 22 or may expose the suction button 22 to an extent that the suction button 22 cannot be removed.

Note that the cover portion 32, at least in a part on at least a side surface of the suction button 22, is preferably flexible or compressible so as not to inhibit the pressing operation of a button top of the suction button 22.

When a top surface of the cover portion 32 and a part of the side surface continuous with the top surface are formed of a rigid material, there is an advantage in that sideway deviation can be prevented. The material of the cover portion 32 is not particularly limited, and the examples of the material may include resin, elastomer, or paper.

When the cover portion 32 is provided with a rigid site, resin, cardboard, metal, or ceramics having predetermined hardness may be attached or welded to the aforementioned material, or when resin is used, only the top surface and the periphery of the top surface may be crosslinked with heat or ultraviolet ray to increase the hardness.

The fixing portion 33 is attached so as to cover an outer periphery of at least one part of the surface of the operation portion 3. As a result, the cover portion 32 is prevented from being deviated from the suction button 22 so as to be fixed without falling off.

Note that the fixing portion 33 is provided so as to cover the surface of the operation portion 3 such that the angle knob 9 as an endoscope handle is exposed to be operatable. The fixing portion 33 is a functional film such as a heat-shrink film, a polypropylene band, or the like.

The fixing portion 33 preferably includes a pressurizing belt that tightens the surface of the operation portion 3 in order to prevent the cover portion 32 from being misaligned from a predetermined site where the cover portion 32 covers the suction button [33]22.

Note that the fixing portion 33 is provided with a fragile region 33a formed of perforations, slits, or the like so that the cover portion 32 is easily removed from the suction button 22 in reprocessing the endoscope 1 at the reprocessing center 102.

In other words, the first reuse prevention tool 31 is detached from the operation portion 3 by cutting off the fixing portion 33 along the fragile region 33a. As a result, the cover portion 32 can be removed from the suction button 22. Therefore, the suction button 22 becomes removable from the suction cylinder 22a of the operation portion 3.

In this manner, in the endoscope 1, the suction button 22 cannot be removed unless the first reuse prevention tool 31 is removed. In other words, the first reuse prevention tool 31 inhibits removal of the suction button 22 from the endoscope 1.

Therefore, in reprocessing the endoscope 1 by the endoscope reprocessor 110, the cleaning tube 105 cannot be attached to the suction cylinder 22a unless the first reuse prevention tool 31 is removed from the endoscope 1.

In the case of handwashing in a sink, a cleaning brush cannot be inserted through the suction cylinder 22a until the first reuse prevention tool 31 is removed.

Note that the first reuse prevention tool 31 may be configured such that the cover portion 32 covers the air/water feeding button 21 as the endoscope accessory. The first reuse prevention tool 31 may also be configured such that the cover portion 32 covers both the suction button 22 and the air/water feeding button 21.

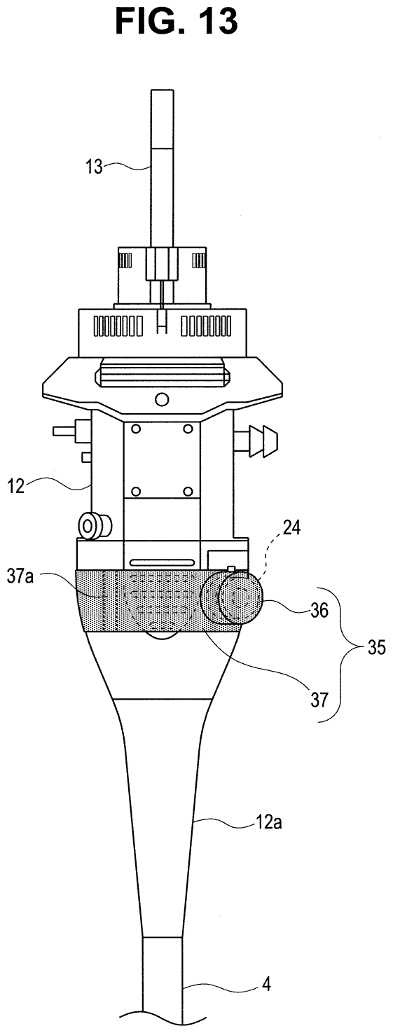

As shown in FIG. 13, a second reuse prevention tool 35 is configured to, for example, cover the ventilation pipe sleeve 24 as the endoscope accessory for water leak testing provided in the scope connector 12.

Specifically, the second reuse prevention tool 35 also includes a cover portion 36 and a fixing portion 37 that fixes the cover portion 36. The cover portion 36 covers the entire ventilation pipe sleeve 24.

The fixing portion 37 is attached so as to cover an outer periphery of at least one part of the surface of the scope connector 12. As a result, the cover portion 36 is prevented from being deviated from the ventilation pipe sleeve 24 so as to be fixed without falling off. The materials of the cover portion 36 and the fixing portion 37 are the same as the material of the aforementioned first reuse prevention tool.

Note that the fixing portion 37 also preferably includes a pressurizing belt that tightens the surface of the scope connector 12 in order to prevent the cover portion 36 from being misaligned from a predetermined site where the cover portion 36 covers the ventilation pipe sleeve 24.

The fixing portion 37 herein is also provided with a fragile region 37a formed of perforations, slits, or the like so that the cover portion 36 is removed from the ventilation pipe sleeve 24 in reprocessing the endoscope 1 at the reprocessing center 102. In other words, the second reuse prevention tool 35 is detached from the scope connector 12 by cutting off the fixing portion 37 along the fragile region 37a.

As a result, the cover portion 36 of the second reuse prevention tool 35 can be removed from the ventilation pipe sleeve 24. Therefore, the ventilation pipe sleeve 24 is exposed in the scope connector 12.

In this manner, in the endoscope 1, the ventilation pipe sleeve 24 is not exposed unless the second reuse prevention tool 35 is removed. In other words, in reprocessing the endoscope 1 by the endoscope reprocessor 110, the air feeding tube 107 cannot be attached to the ventilation pipe sleeve 24 unless the second reuse prevention tool 35 is removed.

When the endoscope reprocessor 110 without an automatic leak tester is used, manual leak testing of the endoscope 1 cannot be performed before performing the reprocessing unless the second reuse prevention tool 35 is removed.

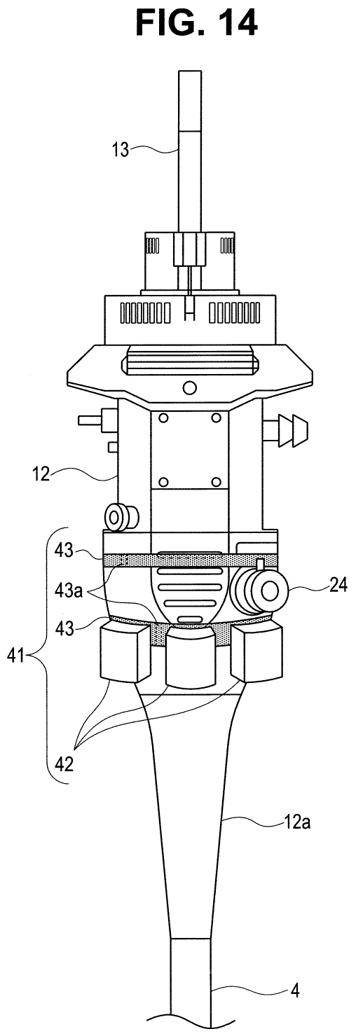

As shown in FIG. 14, a third reuse prevention tool 41 is configured as, for example, a housing prevention tool into the treatment tank 111, which makes the scope connector 12 excessive in size.

Specifically, the third reuse prevention tool 41 includes a plurality of bulks 42 as cover portions and a fixing portion 43 that fixes the plurality of bulks 42. The bulks 42 are rubber pieces, sponges, air-packs, or the like that enlarge the scope connector 12. The plurality of bulks 42 are attached to the fixing portion 43.

The fixing portions 43 is attached so as to cover an outer periphery of at least one part of the surface of the scope connector 12. Note that the fixing portion 43 is provided so as to be wound around the scope connector 12 in such a manner as to sandwich the ventilation pipe sleeve 24 of the endoscope accessory. The fixing portion 43 is also a functional film such as a heat-shrink film, a polypropylene band, or the like.

Note that the fixing portion 43 is also provided with a fragile region 43a formed of perforations, slits, or the like so that the plurality of bulks 42 are removed in reprocessing the endoscope 1. In other words, the third reuse prevention tool 41 is detached from the scope connector 12 by cutting off the fixing portion 43 along the fragile region 43a. As a result, the plurality of bulks 42 of the third reuse prevention tool 41 can be removed from the scope connector 12.

In this manner, in the endoscope 1, the scope connector 12 is excessive in size relative to the treatment tank 111 unless the third reuse prevention tool 41 is removed. In other words, in reprocessing the endoscope 1 by the endoscope reprocessor 110, the scope connector 12 cannot be set at a fixed position on the retaining net 112 of the treatment tank 111 unless the third reuse prevention tool 41 is removed.

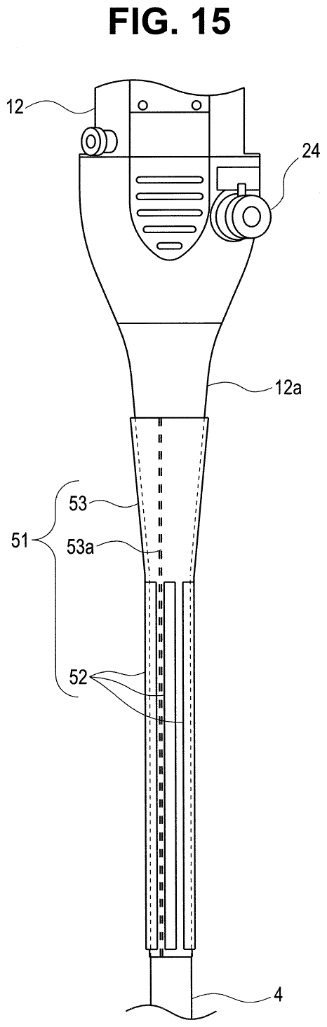

As shown in FIG. 15, a fourth reuse prevention tool 51 is configured as, for example, a bending prevention tool provided from the bend preventing portion 12a of the scope connector 12 halfway through the universal cord 4.

Specifically, the fourth reuse prevention tool 51 also includes a cover portion 52 and a fixing portion 53 that fixes the cover portion 52. The cover portion 52 includes frames which are a plurality of bones having arc-shaped cross-sections covering at least a predetermined length of the universal cord 4 in the longitudinal direction. The plurality of frames of the cover portion 52 are provided at predetermined intervals in a circumferential direction of the universal cord 4.

The cover portion 52 is provided in the fixing portion 53 on the universal cord 4. The cover portion 52 is also formed of rigid synthetic resin or the like having a higher toughness than the universal cord 4. Note that the cover portion 52 preferably includes, on the inner side, a buffer material for preventing damage to the universal cord 4.

The fixing portion 53 covers a predetermined lengthwise range of the universal cord 4 from the bend preventing portion 12a. The fixing portion 53 is a functional film such as a heat-shrink film.

Note that the fixing portion 53 is also provided with a fragile region 53a formed of perforations, slits, or the like so that the cover portion 52 is removed in reprocessing the endoscope 1. In other words, the fourth reuse prevention tool 51 is detached from the bend preventing portion 12a and the universal cord 4 by cutting off the fixing portion 53 along the fragile region 53a. As a result, the cover portion 52 of the fourth reuse prevention tool 51 can be removed from the universal cord 4.

In this manner, in the endoscope 1, bending of the universal cord 4 cannot be performed in a predetermined lengthwise range where the cover portion 52 is provided unless the fourth reuse prevention tool 51 is removed.

In other words, in reprocessing the endoscope 1 by the endoscope reprocessor 110, the universal cord 4 cannot be bent in a predetermined length unless the fourth reuse prevention tool 51 is removed, so that the scope connector 12 or the universal cord 4 cannot be set at a fixed position on the retaining net 112 of the treatment tank 111.

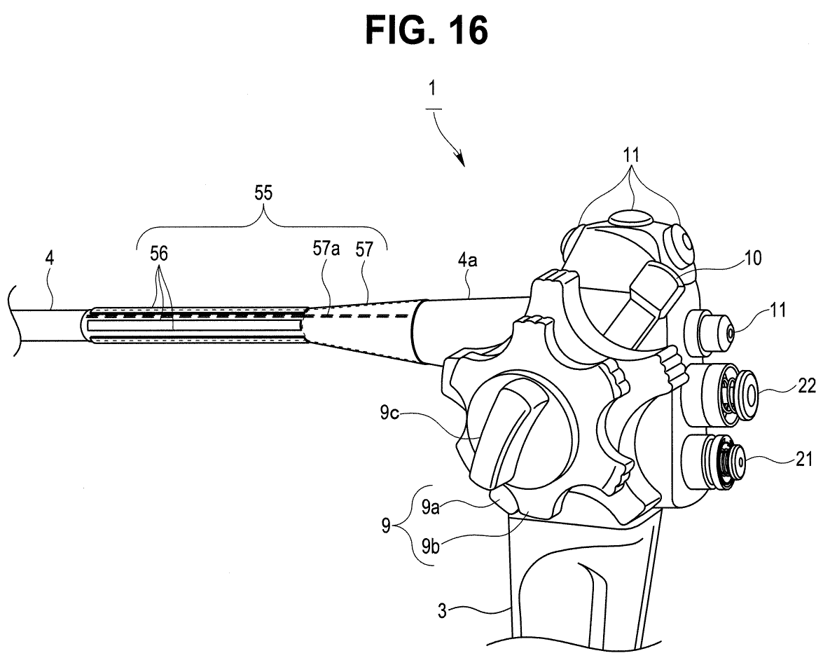

As shown in FIG. 16, a fifth reuse prevention tool 55 is configured as, for example, a bending prevention tool provided from the bend preventing portion 4a of the universal cord 4 of the endoscope accessory halfway through the universal cord 4. The fifth reuse prevention tool 55 has the same configuration as the configuration of the fourth reuse prevention tool 51 and will thus be briefly described.

The fifth reuse prevention tool 55 also includes a cover portion 56 and a fixing portion 57 that fixes the cover portion 56. The fixing portion 57 herein is also provided with a fragile region 57a formed of perforations, slits, or the like so that the cover portion 56 is removed in reprocessing the endoscope 1.

In the endoscope 1 herein also, bending of the universal cord 4 cannot be performed in a predetermined lengthwise range where the cover portion 56 is provided unless the fifth reuse prevention tool 55 is removed.

In other words, in reprocessing the endoscope 1 by the endoscope reprocessor 110, the universal cord 4 cannot be bent in a predetermined shape unless the fifth reuse prevention tool 55 is removed, so that the operation portion 3 or the universal cord 4 cannot be set at a fixed position on the retaining net 112 of the treatment tank 111.

Note that the fourth reuse prevention tool 51 and the fifth reuse prevention tool 55 may be provided halfway in the universal cord 4.

As described above, the endoscope 1 is configured so as not to be reprocessed unless the first to fifth reuse prevention tools 31, 35, 41, 51, and 55 are removed after use. Therefore, the endoscope 1 cannot be reprocessed unless the hospital 101 removes the first to fifth reuse prevention tools 31, 35, 41, 51, and 55 of the used endoscope 1. In this manner, with the first to fifth reuse prevention tools 31, 35, 41, 51, and 55 attached, the reuse of the endoscope 1 by the hospital 101 can be restricted.

Note that the first to fifth reuse prevention tools 31, 35, 41, 51, and 55 are attached to the operation portion 3 or the scope connector 12, while the light guide connector 13 of the scope connector 12, which is connectable to the video processor as an external apparatus, is exposed. In other words, in the endoscope 1 to which the first to fifth reuse prevention tools 31, 35, 41, 51, and 55 are attached, the scope connector 12 is connectable to the video processor.

It is not necessary to attach all the first to fifth reuse prevention tools 31, 35, 41, 51, and 55 to the endoscope 1, and when at least one or more of the first to fifth reuse prevention tools 31, 35, 41, 51, and 55 are attached, the reuse by the hospital 101 can be restricted.

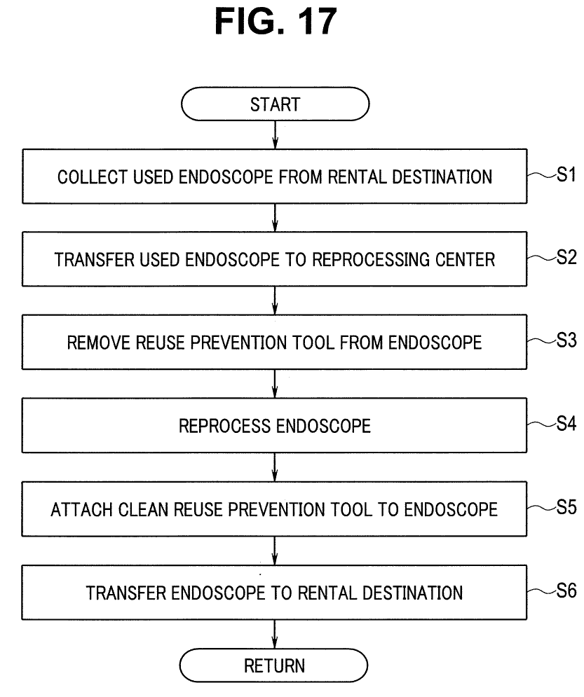

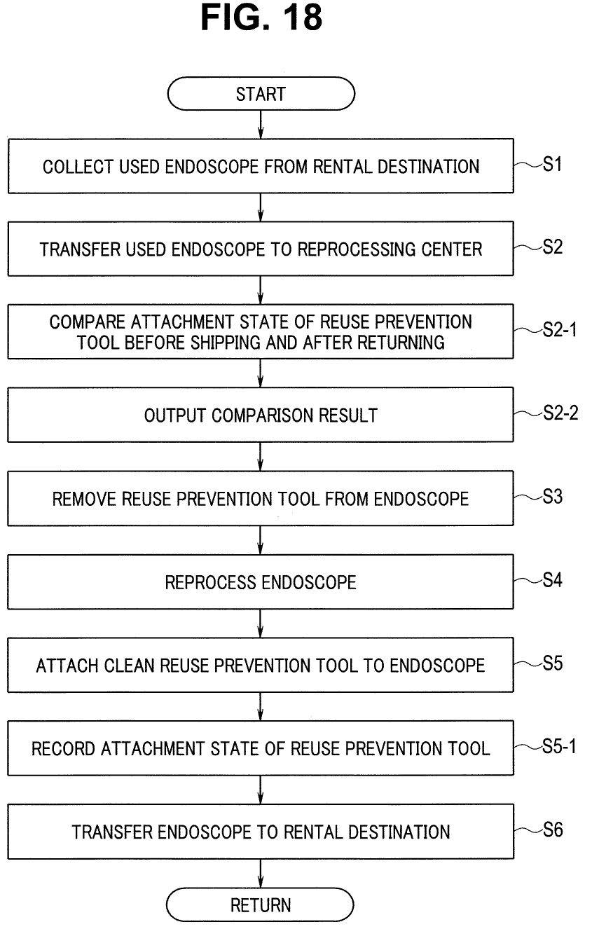

Here, a reprocessing method performed by a rental source company in a rental service of the endoscope 1 will be briefly described based on a flowchart of FIG. 17.

First, a rental source company of the endoscope 1 collects the used endoscope 1 from the hospital 101 (S1). Then, the company transports the used endoscope 1 to the reprocessing center 102 (S2).

The company removes the reuse prevention tools 31, 35, 41, 51, and 55 from the endoscope 1 at the reprocessing center 102 (S3). Then, the company reprocesses the endoscope 1 from which the reuse prevention tools 31, 35, 41, 51, and 55 have been removed (S4).

Next, the company attaches the clean reuse prevention tools 31, 35, 41, 51, and 55 to the reprocessed endoscope 1 (S5). Then, the company transports the endoscope 1 to which the reuse prevention tools 31, 35, 41, 51, and 55 are attached to the rental destination (S6).

In this manner, the company as the rental source of the endoscope 1 reprocesses the used endoscope 1 that has been rented out to the hospital 101, and rents out the reprocessed endoscope 1 to the hospital 101 again.

Note that the company confirms whether the reuse prevention tools 31, 35, 41, 51, and 55 are properly attached, before reprocessing the used endoscope 1 at the reprocessing center 102, and if the reuse prevention tools 31, 35, 41, 51, and 55 are removed, a possibility that the hospital 101 has reused the endoscope 1 can be recognized.

Thus, the possibility that the hospital 101 has reused the endoscope 1 can be easily determined and the responsibility for quality management lying between the rental destination and the rental source can be clarified.

Note that as a modification of the reprocessing method performed by the rental source company in the rental service of the endoscope 1, as shown in FIG. 18, a step of comparing attachment states of the reuse prevention tools before shipping and after returning may be added as a reprocessing confirmation method. This makes it possible to more precisely determine whether the reuse prevention tools have been misaligned or returned to the original positions after misalignment or removal.

As a method for realizing the present modification, the confirmation may be made such that the reuse prevention tools are recorded before shipping as a photograph or a moving image and after returning, a person compares the photograph or the moving image and the state of the reuse prevention tools of the endoscope at hand or the comparison may be made using an apparatus.

When the apparatus is used, a processor including a recording section having a recording function and a comparing section having a comparing function is used, and the recording section records the attachment state of the reuse prevention tools of the endoscope before shipping to which the reuse prevention tools are attached (S5-1).

Next, after the endoscope is returned, the comparing section compares the attachment state of the reuse prevention tools with the attachment state recorded, before removing the reuse prevention tools for the reprocessing (S2-1) and further outputs the comparison result (S2-2).

Next, the recording section records the attachment state of the reuse prevention tools of the endoscope before shipping to which the reuse prevention tools are newly attached after reprocessing (S5-1). As the comparing method, a publicly-known image processing method or the like may be appropriately used. The processor may be mounted on the endoscope reprocessor or may be an apparatus separate from the endoscope reprocessor.

As described above, the reprocessing (confirmation) method for the endoscope 1, the reuse prevention tools 31, 35, 41, 51, and 55, and the endoscope 1 of the present embodiment enable the rental source company to easily determine whether the endoscope 1 has been reprocessed by the hospital 101 or the like, and make it possible to clarify the responsibility for quality management lying between the rental destination and the rental source.

The disclosure described in the aforementioned embodiment and modifications is not limited to the embodiment and modifications, and can be implemented with various modifications within the scope without departing from the gist of the disclosure at the implementation stage. Further, the aforementioned embodiment and modifications encompass the disclosures at various stages, and various disclosures can be extracted by appropriate combinations of a plurality of components disclosed herein.

For example, even if some components from the components described in the embodiment and modifications are deleted, if the problem stated herein can be solved, and if the advantageous effects stated herein can be obtained, the configuration without the deleted components can be extracted as a disclosure.

What is claimed is:

1. A reprocessing confirmation method for an endoscope, wherein
a processor is configured to:
record an attachment state of a reuse prevention tool of the endoscope before shipping to which the reuse prevention tool is attached;
after the endoscope is returned, compare the attachment state of the reuse prevention tool with the attachment state recorded, before removing the reuse prevention tool for reprocessing and further output a comparison result; and
record the attachment state of the reuse prevention tool of the endoscope before shipping to which the reuse prevention tool is newly attached after reprocessing.

2. A reuse prevention tool comprising:
a cover that covers at least one part of an endoscope accessory or an endoscope main body portion on an endoscope surface, with a light guide exposed so as to be connectable to an endoscope processor; and
a fixing portion that fixes the cover to the endoscope surface.

3. The reuse prevention tool according to claim 2, wherein the cover inhibits removal of the endoscope accessory from the endoscope or prevents the endoscope from being reprocessed, by making the endoscope excessive in size relative to a treatment tank of an endoscope reprocessor.

4. The reuse prevention tool according to claim 2, wherein the cover exposes an endoscope handle so that the endoscope handle is operable.

5. The reuse prevention tool according to claim 2, wherein when the cover covers the endoscope accessory, the endoscope accessory is a suction button, an air/water feeding button, or a ventilation pipe sleeve.

6. The reuse prevention tool according to claim 2, wherein
when the cover covers the at least one part of the endoscope main body portion, the at least one part is a universal cord, and
the cover comprises a bone having a higher toughness than the universal cord and having a predetermined length along the universal cord.

7. The reuse prevention tool according to claim 2, wherein
when the cover covers the at least one part of the endoscope main body portion, the at least one part is a scope connector, and
the cover comprises a bulk that enlarges an outer periphery of the scope connector when the cover is attached.

8. The reuse prevention tool according to claim 2, wherein the fixing portion comprises a pressurizing belt configured to prevent the cover from being misaligned from a predetermined site.

9. The reuse prevention tool according to claim 8, wherein the pressurizing belt is a heat-shrink film.

10. The reuse prevention tool according to claim 2, wherein the fixing portion comprises a fragile region that is more fragile than an adjacent region.

11. The reuse prevention tool according to claim 2, comprising an RF tag.

12. An endoscope comprising the reuse prevention tool according to claim 2, wherein the reuse prevention tool is disposed on an endoscope main body, the endoscope accessory, and a surface of the endoscope accessory or the endoscope main body portion.

13. The endoscope according to claim 12, wherein the cover inhibits removal of the endoscope accessory from the endoscope or prevents the endoscope from being reprocessed, by making the endoscope excessive in size relative to a treatment tank of an endoscope reprocessor.

14. The endoscope according to claim 12, wherein the cover exposes an endoscope handle so that the endoscope handle is operable.

15. The endoscope according to claim 12, wherein when the cover covers the endoscope accessory, the endoscope accessory is a suction button, an air/water feeding button, or a ventilation pipe sleeve.

16. The endoscope according to claim 12, comprising an RF tag.
